(19) United States
(12) Patent Application Publication
(10) Pub. No.: US 2013/0295036 A1
(43) Pub. Date: Nov. 7, 2013

(54) METHOD OF USING WOOD EXTRACTS IN COSMETIC AND HYGIENE PRODUCTS

(71) Applicants: Sergey V. Philippov, Moscow (RU); Igor M. Bogorodov, Moscow (RU)

(72) Inventors: Sergey V. Philippov, Moscow (RU); Igor M. Bogorodov, Moscow (RU)

(73) Assignee: FLAVITPURE, INC., Cheyenne, WY (US)

(21) Appl. No.: 13/464,983

(22) Filed: May 5, 2012

(51) Int. Cl. A61K 8/97; A61Q 19/00; A61Q 5/00

(52) U.S. Cl. 424/74; 424/770

(57) ABSTRACT

A method of using wood extracts as natural compounds in cosmetic and hygiene products. In particular, the method includes using natural compounds derived from a plant material wherein the compounds are at least one of antioxidant Dihydroquercetin (taxifolin), polysaccharide Arabinogalactan, Arabinogalactan combined with Dihydroquercetin (taxifolin) and wood oleoresin comprising oil and resin, and wherein all these natural compounds are consequently extracted from wood and\or by-products of logging industry.

### REFERENCES

[1] Fitzpatrick A., Roberts A and Witherly S. (2004). Larch Arabinogalactan: a novel and multifunctional natural product. Agro Food industry Hi-Tech 15(1):30-32.
[2]Ohr L. M. Arabinogalactan Adds More than Health Benefits//Prepared Foods. 2001. V. 170. No1.P. 55.
[3]Tatjana Stevanovic, Papa NiokhorDiouf and Martha Estrella Garcia-Perez (2009). Bioactive Polyphenols from Healthy Diets and Forest Biomass. Current Nutrition & Food Science. Vol. 5. No. 4, p. 264-295.
[4] Lee S B, Cha K H, Selenge D. Solongo A. Nho C W (2007). The chemopreventive effect of taxifolin is exerted through ARE-dependent gene regulation. Biol Pharm Bull., 30(6): 1074-1079.
[5] Gupta M B. Bhalla T N, Gupta G P, Mitra C R, Bhargava K P. (1971). Anti-inflammatory activity of taxifolin. Japan J Pharmacol., 21 (3):377-82.
[6]V. I. Bol'shakova, V. A. Khan, Zh. V. Dubovenko, E. N. Shmidt, and V. A. Pentegova. TERPENOIDS OF THE OLEORESIN OF THE LARCH GROWING IN KAMCHATKA. Novosibirsk Institute of Organic Chemistry. Siberian Branch, Academy of Sciences of the USSR. Translated from Khimiya Prirodnykh Soedinenii, No. 3. pp. 340-343, May-June, 1980.© 1980 Plenum Publishing Corporation.
[7]G. F. Chernenko and E. N. Shmidt. NEUTRAL EXTRACTIVE SUBSTANCES FROM THE BARK OF Larixsibirica, Novosibirsk Institute of Organic Chemistry. Siberian Branch, Academy of Sciences of the USSR. Translated from Khimiya Prirodnykh Soedinenii, No. 6. pp. 833-834. November-December, 1990.© 1991 Plenum Publishing Corporation.
[8]L. G. D'yachenko, V. I. Roshchin, and V. E. Kovalev. NEUTRAL COMPOUNDS OF THE EXTRACTIVE SUBSTANCES OF Larixgmelini. S. M. Kirov Academy of Wood Technolog˜Leningrad. Translated from Khimiya Prirodnykh Soedinenii, No. i, pp. 56-63, January-February, 1986.© 1986 Plenum Publishing Corporation.
[9]Lisina, A. I., L. N. Volskil, Y. G. Leont'eva and V. A. Pentegova. (1969). Extractives of the heartwood and sapwood of Larixgmelini. Izu. Sib. Otd. Akad. Nauk SSSR, Ser. Khim. Nauk. 6:10.2-104 (not seen, Chem. Abstr. 72;68404b).
[10]BITO T; ROY S; SEN C K; SHIRAKAWA T; GOTOH A; UEDA M; ICHIHASHI M; PACKER L. Flavonoids differentially regulate IFN-gamma-induced ICAM-1 expression in human keratinocytes; molecular mechanisms of action. FEBS Lett., 2002, vol. 520 (1-3), 145-52.
[11]Patrick Kramer, (2010). Taxifolin: A Trendy Yet Trustworthy Cosmetics Compound. Presentation Abstracts, CHEM 23201.
[12]MIDDLETON E; KANDASWAMI C; THEOHARIDES T C. The effects of plant flavonoids on mammalian cells: implications for inflammation, heart disease and cancer. Pharmacol Rev, 2000, vol. 52 (4), 673-752.
[13]Young Ahn, Sun Eun Choi, MiSook Jeong, Kwan Hee Park, Nam Ju Moon, Seong Soo Joo, Chung Soo Lee, Young Wook Choi, Kapsok Li, Mi-Kyung Lee, Min Won Lee, Seong Jun Seo. (2009). Effect of taxifolin glycoside on atopic dermatitis-like skin lesions in NC/Nga mice. Phytotherapy Research, Published Online: 29 Dec 2009.
[14]Harlinda Kuspradini, Tohru Mitsunaga and Hideo Ohashi.(2009). Antimicrobial activity against Streptococcus sobrinus and glucosyltransferase inhibitory activity of taxifolin and some flavanonolrhamnosides from kempas (Koompassiamalaccensis) extracts. Journal of Wood Science, Volume 55, Number 4, Pages 308-313.
[15]Haraguchi H, Mochida Y, Sakai S. Masuda H, Tamura Y, Mizutani K, Tanaka O, Chou W H. (1996). Protection against oxidative damage by dihydroflavonols in Engelhardtiachrysolepis.BiosciBiotechnolBiochem., 60(6):945-8.)
[16]Kostyuk V A, Potapovich A I. (1998). Antiradical and chelating effects in flavonoid protection against silica-induced cell injury. Arch Biochem Biophys., 355(1):43-8.
[17] Godley, Bernard F. and Shamsi, FarrukhAnis and Liang, Fong-Qi and Jarrett, Stuart Gordon and Davies, Sallyanne and Boulton, Michael Edwin. (2005). Blue light induces mitochondrial DNA damage and free radical production in epithelial cells. Journal of Biological Chemistry, 280 (22). pp. 21061-21066. ISSN 00219258.
[18]Xinyu JIANG, Xiaoqing CHEN * and Yan WEI.(2009). Free Radical Scavenging Activity and Flavonoids Contents of Polygonumorientale Leaf, Stem and Seed Extracts. Lat. Am. J. Pharm. 28 (2): 284-7.
[19]Iskandarov, A. I., Abdukarimov, B. A. (2009). Influence of Dihydroquercetin and ascorbic acid on the content of malondialdehyde and metallothionein in rat's organs exposed to chronic cadmium impact. Journal Toxicological Vestnik, volume 4. Russian language version.
[20]Yifan Chen. (2009). Antioxidants quercetin and dihydroquercetin inhibit ex vivo hemolysis but not plasma lipid peroxidation. FASEB J. 23: 966.3.
[21]Bronnikov, G. E., Kulagina, T. P., Aripovsky, A. V. (2009). Dietary Supplementation of Old Mice with Flavonoid Dihydroquercetin Causes Recovery of Mitochondrial Enzyme Activities in Skeletal Muscles. ISSN 1990-7478, Biochemistry (Moscow) Supplement Series A: Membrane and Cell Biology. 2009, Vol. 3, No. 4, pp. 453-458. Pleiades Publishing, Ltd. Russian language version. Original Russian Text © G. E. Bronnikov, T. P. Kulagina, A. V. Aripovsky. 2009, published in BiologicheskieMembrany. 2009, Vol. 26. No. 5. pp. 387-393. Russian language version.
[22]S. Panyushin, B. Sakharov, S. Chubatova, O. Bolshakova. (1999). Sun defensive and anti-radical properties of plant biological actives. Cosmetic & Medicine, (5-6), Issue 56. Russian language version.
[23]2005 “Cosmetics & Medicine”, Russian Journal.
[24]VAN ACKER SABE, VAN DEN BERG D J, TROMP M N J L, GRIFFIOEN D H G, VAN BENNEKOM, VAN DER V I J G H W J F. BAST A (1996) Structural aspects of antioxidant activity of flavonoids. Free RadicBiol Med 20:331-342.
[25]van der L B, Bachschmid M. Spitzer V, et al. Decreased plasma and tissue levels of vitamin C in a rat model of aging: implications for antioxidative defense. Biochem Biophys Res Commun. 2003 Apr. 4;303(2):483-7.
[26]Potapovich A I, Kostyuk V A. Comparative study of antioxidant properties and cytoprotective activity of flavonoids. Biochemistry (Most.). 2003 May;68(5):514-9.
[27]Kravchenko L V, Morozov S V. Tutel'van V A. Effects of flavonoids on the resistance of microsomes to lipid peroxidation in vitro and ex vivo. Bull ExpBiol Med. 2003 Dec; 136(6):572-5.
[28]Teselkin Y O, Babenkova I V, Tjukavkina N A, et al. Influence of dihydroquercetin on the lipid peroxidation of mice during postradiation period. Phytotherapy Research. 1998;12:517-9.
[29]Vasiljeva O V, Lyubitsky O B, Klebanov G I, Vladimirov Y A. Effect of the combined action of flavonoids, ascorbate and alphatocopherol on peroxidation of phospholipid liposomes induced by Fe2+ ions. Membr Cell Biol. 2000;14(1):47-56.
[30] Li. Y., and Jaiswal. A. K. (1994) Human antioxidant-response-element mediated regulation of type 1 AD(P)H:quinoneoxidoreductase gene expression. Eur. J. Biochem., 226. 31-39, 1994.
[31]Saet Byoul Lee, Kwang Hyun Cha, Dangaa Selenge, Amgalan Solongo, and Chu Won Nho. (2007). The Chemopreventive Effect of Taxifolin Is Exerted through ARE-Dependent Gene Regulation. Biol. Pharm. Bull., 30(6) 1074-1079.
[32]Crespo I., Garcia-Mediavilla M. V., Almar M. et al. 2008.
[33]Ishige K. Schubert D, Sagara Y. Flavonoids protect neuronal cells from oxidative stress by three distinct mechanisms. Free RadicBiol Med. 2001:30:433-446.
[34]Kolhir, V. K., et.al. 1996. Antioxidant activity of a dihydroquercetin isolated from Larixgmelinii. Phytotherapy Research. 10(6): 478-482.
[35]Ivanenkov, Y A; Balakin, K V; Tkachenko, S E. (2008). New Approaches to the Treatment of Inflammatory Disease: Focus on Small-Molecule Inhibitors of Signal Transduction Pathways. Drugs in R & D, Volume 9—Issue 6—pp 397-434.
[36]Xin-Xin Zhang, Xue-Feng Xiao, Qi Shan, Wen-Bin Hou. (2010). Recent Advance on Plant Sources, Bioactivities, Pharmacological effects and Pharmacokinetic Studies of Taxifolin, Asian Journal of Pharmacodynamics and Pharmacokinetics. 10(1):35-43.
[37] Devi M A, Das N P. In vitro effects of natural plant polyphenols on the proliferation of normal and abnormal human lymphocytes and their secretions of interleukin-2.Cancer Lett. 1993 May 14;69(3):191-6.
[38] Kim Y J, Choi S E, Lee M W. Lee C S. (2008). Dihydroquercetin (taxifolin) inhibits dendritic cell responses stimulated by lipopolysaccharide and lipoteichoic acid. J Pharm Pharmacol. 60(11):1465-72.
[39] Sang Mi An, Hyo Jung Kim, Jung-Eun Kim, Yong Chool Boo. (2008). Flavonoids, taxifolin and luteolin attenuate cellular melanogenesis despite increasing tyrosinase protein levels. Phytotherapy Research. Volume 22, Issue 9, Pages 1200-1207.
[40]Bjeldanes L F, Chang G W. Mutagenic activity of quercetin and related compounds. Science. 1977 Aug 5;197(4303):577-8.
[41] Nagao M, Morita N, Yahagi T, et al. Mutagenicities of 61 flavonoids and 11 related compounds. Environ Mutagen. 1981;3 (4):401-19.
[42] Booth A N, Deeds F. The toxicity and metabolism of dihydroquercetin. J Am Pharm Assoc Am Pharm Assoc (Baltim.). 1958 Mar:47(3, Part 1):183-4.
[43] William S. Branham, Stacey L. Dial, Carrie L. Moland, Bruce S. Hass, Robert M. Blair, Hong Fang, Leming Shi, Weida Tong. Roger G. Perkins and Daniel M. Sheehan. (2002). Phytoestrogens and Mycoestrogens Bind to the Rat Uterine Estrogen Receptor. Biochemical and Molecular Action of Nutrients, 2002 American Society for Nutritional Sciences.
[44] Wendy N. Jefferson, Elizabeth Padilla-Banks, George Clarkb, Retha R. Newbold. (2002). Assessing estrogenic activity of phytochemicals using transcriptional activation and immature mouse uterotrophic responses. Journal of Chromatography B, 777, pp. 179-189.
[45]Wim Watjen, Gudrun Michels, BarbelSteffan, Petra Niering, YvonniChovolou, Andreas Kampkotter, Quynh-Hoa Tran-Thi, Peter Proksch, and RegineKahl. (2005). Low Concentrations of Flavonoids Are Protective in Rat H4IIE Cells Whereas High Concentrations Cause DNA Damage and Apoptosis J. Nutr. 135: 525-531.
[46] Kathrin Plochmanna, Gabriele Korte, EleniKoutsilieri. ElkeRichling, Peter Riederer, Axel Rethwilm, Peter Schreier and CarstenScheller. (2007). Structure-activity relationships of flavonoid-induced cytotoxicity on human leukemia cells. Archives of Biochemistry and Biophysics. Volume 460. Issue 1. Pages 1-9.
[47]Stavreva, M., et al. (2008). Protocol on Toxicological Investigations and Safety Evaluation of DHQ for application in food products, National Center of Public Health and Nutrition, Director Ivanov, L., Ministry of Health, Sofia, Bulgaria. Agreement No. 034-P-2007. Bulgarian language version.
[48]Zhanataev, A. K., Kulakova, A. V., Nasonova, V. V., Durnev, A. D., (2008). In Vivo Study of Dihydroquercetin Genotoxicity. Bulletin of Experimental Biology and Medicine, 145, 3, 309-312. PHARMACOLOGY AND TOXICOLOGY.
[49]Makena, Patrudu S; Pierce. Samuel C; Chung, King-Thom; Sinclair, Scott E; (2009). Comparative mutagenic effects of structurally similar flavonoids quercetin and taxifolin on tester strains Salmonella typhimurium TA 102 and Escherichia coli WP-2 uvrA. Environmental and molecular mutagenesis (Environ Mol Mutagen), vol. 50 (issue 6): pp. 451-9.
[50] Terziev, N. (2002b): Properties and Processing of Larch Timber—a Review based on Soviet and Russian Literature.
[51]Westman M. 1999. Galactoarabinan: An Exfoliant for Human Skin, Cosmetics and Toiletries 114 8. 63-72.
[52]K. Dimas et al. Planta Med. 1998, 208-21 1; K. Dimas et al. Leukemia Res. 1999, 217-234; K. Dimas et al. Anticancer Res. 1999, 4065-4072.
[53]Itokawa, H. et all. Planta Med. 1988, 311-315; K. Dimas et al. Planta Med. 1998, 208-211; K. Dimas et al. Leukemia Res. 1999, 217-234; K. Dimas et al. Anticancer Res. 1999, 4065-4072.
[54]Sonia Savluchinske-Feio, Maria João Marcelo Curto, Bárbara Gigante and J. Carlos Roseiro. (2006) APPLIED MICROBIOLOGY AND BIOTECHNOLOGY Volume 72, Number 3, 430-436.
Additionally, Applicant incorporates herein by reference the following patent documents:
EU patent No. EP 0939771B1. RICHARDS, Geoffrey N., Water soluble lipidated arabinogalactan.
U.S. Pat. No. 6,406,686B1. Ho-Ming Chun. CONDITIONING SHAMPOO CONTAINING ARABINOGALACTAN.
U.S. Patent No. 2011/10123471A1. Jatinder Rana et al., TOPICAL COMPOSITION WITH SKIN LIGHTENING EFFECT.
U.S. Patent No. 2009/0317342A1. Thomas Rudolph et al., USE OF FLAVONOIDS.
DE Patent application No. 20080220031. Thomas Wunsch et al., Dermocosmetic Preparations.
WO/2009/134165 Tikhonov et al., ANTIOXIDANT AND ANTIHYPOXANT DIHYDROQUERCETIN-BASED COMPLEX FOR COSMETIC PRODUCTS.
U.S. Pat. No. 5,641,489 Fleisher., Extracting maltol and water from naturally occurring plant material containing maltol and water.
U.S. Patent No. 2006/0140881A1. GuofengXu et al., ORAL CARE COMPOSITIONS CONTAININGFLAVONOIDS AND FLAVANS.
WO 2009/079680 A1. BAUER. Rudolf et al., USE LARCH WOOD MATERIAL FOR TREATING INFLAMMATION.
U.S. Pat. No. 3,943,248, Shulman, Max J. Methods of treating burns using colophony containing preparations.

### FIELD OF THE INVENTION

The present invention is directed to the use of wood extracts or natural compounds for applications in cosmetic and hygiene products.

### BACKGROUND OF THE INVENTION

Wood extracts have been applied in medicine, pharmacy and skin care since the ancient times. Extracts for cosmetic use must be aesthetic, acceptable in terms of odor and color and, moreover, free from toxic chemicals. The present invention is primarily focused on the exploitation of wood extracts from residues of Conifer wood species, especially those from the family of Pinaceae. The emphasis is put on residues of wood transformation such as bark, butt logs, roots and knot wood, which are considered to be waste products in wood industry. At present these residues are mainly used as a fuel material. The material is cheap and easily available in high amounts. As this material comprises waste products in wood industry, the exploitation of wood extracts from this material for cosmetic and hygiene care purposes would significantly enhance its value.

The plant genus Larix refers generally to any of the numerous conifers in the family of Pinaceae that have deciduous needlelike leaves. Larch wood is known to contain lignans, flavonoids, polysaccharides and oleoresin. Applications for larch wood extracts, in particular the polysaccharide Arabinogalactan and flavonoid Dihydroquercetin (taxifolin), are found in the food, pharmaceutical and cosmetic industries. Larch arabinogalactan, a water-soluble polysaccharide deriving mainly from plant genus Larix, is the source of dietary fiber, but has also confirmed effects as prebiotic [1,2]. The flavonoid constituents of larch wood in particular the flavonoid Dihydroquercetin (taxifolin) is known to possess good antioxidant and anti-inflammatory activities [3-5]. The oleoresins of the coniferous trees are well known in the flavor, fragrance, cosmetic and pharmaceutical industries. At the present time, the oleoresins of various species of larch have been studied in detail to consider actual content of biological active natural compounds [6-8]. There is information on the composition of the oleoresinterpenoids as extractive substances of the trunk part of the European larch and the heart and sapwood and the bark of the Larixcajanderi, Larixczekanowskii, Larixdahurica, Larixgmelinii, Larixkamtschatica, Larixrussica, Larixsibirica, Larixsukaczewii. The neutral substances composing 50% of the weight of the larch oleoresin were represented by hydrocarbons and oxygen-containing compounds deterpenes (16% and 34%, respectively). Hydrocarbons are presented by monoterpene hydrocarbons, sesquiterpenes and diterpene hydrocarbons and aldehydes. The main components of the neutral substances are bicyclic compounds deterpenes or deterpenoids with the labdane structure: epimanool (˜15%), and larixol (˜40%) and its monoacetate (larixylacetate ˜28%), making up about one-third of the neutral substances. In the acidic fraction of the oleoresin. isopimaric acid (40%) predominates [9]. Diterpenoids are constituents of natural resins, such as colophony resin, which is gained from conifer trees like spruce, firs and pines. Larches belong to the family Pinaceae, as already mentioned.

According to the present invention “wood extracts” refers to all kind of extractable raw wood material obtained from a tree of the genus Larix. Preferably the wood extracts are obtained from extractable raw wood material used from Larixcajanderi, Larixczekanowskii, Larixdahurica, Larixgmelinii, Larixkamtschatica, Larixrussica, Larixsibirica, Larixsukaczewii. The larch wood material can, however, be derived from other members of the genus Larix as well. Preferably the extractable larch wood material is larch sawdust, which is a waste product in wood industry. It is inexpensive and easily available in large amounts. The term “larch sawdust” also refers to larch wood shavings. Other kinds of waste wood from larch (e.g. bark, wastes accruing in woodcutting, scrap wood) can also be used within the frame of the present invention.

According to the present invention cosmetics and hygiene products shall mean any substance or preparation intended to he placed in contact with the external parts of the human body or with the teeth and the mucous membranes of the oral cavity with a view exclusively or mainly to cleaning or perfuming them, changing their appearance, correcting body odors, protecting them, or keeping them in good condition.

Cosmetic, including hygiene care is a global billion-dollar industry that markets and sells beauty and healthy products. However, environmental and health worries associated with manufactured goods undermine consumer confidence. Larch wood extracts flavonoid Dihydroquercetin (taxifolin), polysaccharide Arabinogalactan and Arabinogalactan combined with Dihydroquercetin (taxifolin) and wood oleoresin comprising oil and resin are presented potential to solve these concerns. In this invention, the syntheses, characterization, and other useful chemical properties of larch wood extracts are analyzed. In this invention, scientific literature concerning larch wood extracts is scrutinized to evaluate their environmental impact and chemical contribution to cosmetic and hygiene products.

The skin is one of the most important organs of the body and creates a first line of organism defense against the external environment. Owing to the constantly increasing requirement for cosmetic and hygiene active natural compounds, it is the object of the present invention to identify active larch wood extracts which (i) have as little irritation potential as possible for the skin, hair, oral cavity (ii) have a high free radical-deactivating and anti-inflammatory effects and (iii) have a potential of bioavailability-enhancing agents(iv) are also suitable for the preparation of cosmetic and/or hygiene formulations or preparations. In particular, it was the object of the present invention to provide active larch wood extracts and compositions comprising these active extracts or natural compounds, which active compounds and compositions have a free radical-decomposing effect and can be used for avoiding or reducing skin and/or hair damage caused by said free radicals. It was furthermore of particular interest to identify active compounds which stabilize oxidation-sensitive cosmetically and hygiene active substances and prolong the stability of such formulations. It was furthermore also an object of the invention to identify active compounds which possess strong anti-inflammatory activity in cosmetic and hygiene compositions. In particular, it was an object of the present invention to identify natural compounds which are suitable for the use in cosmetic and hygiene formulations or compositions as bioavailability-enhancing agents that includes a solubilizing agents and/or surface active agents, which generally are an important aspect of the cosmetic and hygiene compositions, as they can function as stabilizers, surfactants, emulsifiers, foam modulators, and/or active ingredient dispersion agents. It was also the object of present invention to identify active natural compounds to provide many desirable skin, hair, oral care effects.

EP 0939771B1 discloses lipidated arabinogalactan compositions for a wide variety of different biomedical applications including the inhibition of infection or inflammation.

U.S. Pat. No. 6,406,686B1 discloses use of the Arabinogalactan to provide a stable suspension of silicone in a shampoo even where that shampoo has a low viscosity.

U.S. 2011/10123471A1 discloses topical compositions to provide skin whitening or lightening effect that contains the combination of a pomegranate extract and an extract derived from the plant genus Larix standardized to about 80% taxifolin.

U.S. 2009/0317342A1 discloses the use of at least one flavonoid for odor improvement and/or odor stabilization, and to corresponding compositions and the preparation thereof, wherein particular preference was given to flavonoids as rutin sulfate and troxerutin.

DE 20080220031 discloses the use of flavones in dermocosmetic formulations and the use of the dermocosmetics according to the invention for reducing or avoiding skin or hair damage caused by free radicals.

WO/2009/134165 discloses the use antioxidant and anti-hypoxant dihydroquercetin-phospholipid based complex for cosmetic products.

U.S. 2006/0140881A1 discloses oral composition comprising a free-B-ring flavonoid, a flavan, and a bioavailability-enhancing agent for increasing the bioavailability of the flavonoid and/or the flavan in the oral tissue in the oral cavity.

DE 3113460A1 discloses a pharmaceutical composition for treatment of chronic disorders such as arthritis and rheumatism, wherein the preparation contains a mixture of resin acids. including the diterpenes, laevopimaric acid, neoabietic acid, palustricacid, abietic acid, pimaric acid and isopimaric acid. The composition is most effective when applied topically, but might also be applied orally.

WO 2009/079680 A1 discloses the use of raw larch wood material as a medicament and an anti-inflammatory food/feed supplement for animals and humans.

### BRIEF SUMMARY

The present invention addresses some of the above issues. In one aspect of the invention, the wood extract in particular Dihydroquercetin (taxifolin) is provided for reducing or inhibiting free radical oxidative damage, harmful microbial and inflammatory effects, thus cosmetic and/or hygiene compositions include an effective amount of Dihydroquercetin (taxifolin) extract. The wood extract may be standardized to about 80% Dihydroquercetin (taxifolin).

Another aspect of the invention includes a cosmetic and hygiene compositions that contain the combination of a Dihydroquercetin (taxifolin) and Arabinogalactan as one extract to reduce or inhibit free radical oxidative damage, harmful microbial and inflammatory effects resulting in skin, hair and oral care benefits. In one aspect, the polysaccharide Arabinogalactan can be defined as a fiber containing significant amounts of natural antioxidants, mainly Dihydroquercetin (taxifolin) associated naturally to the fiber matrix with the following specific characteristics: 1. Fiber content, higher than 70% dry matter basis, 2. One gram of fiber Arabinogalactan should have a capacity to inhibit lipid oxidation equivalent to, at least, 1,000 umol TE/gram basing on ORAC value and normally to 2,000-4,000 umol TE/gram 3. One gram of fiber Arabinogalactan should have a capacity of Cell-based Antioxidant Protection (CAP-e) to protect live cells from oxidative damage to, at least 6 CAP-e units per gram. where the CAP-e value is in Gallic Acid Equivalent (GAE) units. 4. The antioxidant capacity must be an intrinsic property, derived from natural constituents of the material not by added antioxidants or by previous chemical or enzymatic treatments. It has been found that surprisingly and unexpectedly the combination of Arabinogalactan and Dihydroquercetin (taxifolin) synergistically can effectively be used for oral and topical applications and serve as a natural pool of nutrients and growth factors that support skin and oral health.

Another aspect of the invention includes a cosmetic and hygiene compositions that contain Arabinogalactan extract, a suitable polymer, nonionic, water-soluble or water-dispersible polymer to provide support to skin texture and hydration and preserve skin elasticity and considered to be bioavailability-enhancing and surface active agent, which can function as surfactant, emulsifier, foam modulator, and/or active ingredient dispersion agent. In one aspect, the Arabinogalactan can be defined as a fiber containing significant amounts of natural antioxidants, mainly Dihydroquercetin (taxifolin) associated naturally to the fiber matrix as already described above and to enjoy a special status in the repository of cosmetic and hygiene ingredients that nurture skin and oral health and wellbeing.

Another aspect of the invention includes a cosmetic and hygiene compositions that contain wood oleoresin extract in forms of oil and/or resin an another class of natural actives that support skin and oral health comprising neutral part of oil and resin especially derived from wood oleoresin for application in cosmetic and hygiene preparations or formulations in order to provide active role in limiting hair loss, stimulating skin pigmentation, and preserving skin or oral health, to provide the body's early defense in response to trauma, inflammation or infection, the acute phase response (APR), which is a complex set of systemic reactions seen shortly after exposure to a triggering event. In one aspect, the oleoresin can be combined with Dihydroquercetin (taxifolin) naturally as one extract or syntactically, wherein Dihydroquercetin (taxifolin) might be in content of 0.1% to 30%.

For the purposes of the present invention, the terms preparation and formulation are also used synonymously in addition to the term composition.

The wood extracts or natural compounds and/or their derivatives and/or mixture of natural compounds and their derivatives may be applied to the cosmetic or hygiene composition by mixing them in preparation so that natural compounds are retained with the composition in an amount effective to achieve above objects or purposes of present invention. Alternatively, the natural compounds and/or their derivatives and/or mixture of natural compounds and their derivatives can be applied using a technique selected from the group consisting of spraying, dipping, rinsing, brushing, or a combination thereof.

The object of the present invention was accordingly to find ways of stabilizing or improving the cosmetic or hygiene compositions or premixes, to enhance the keeping quality or stability of a compositions or to improve its organoleptic properties, without change the nature, substance or quality of the composition and to provide aids in manufacture, processing, preparation, treatment, packing, transport or storage of compositions.

Surprisingly, it has now been found that this is achieved by the use of at least one natural compound, preferably by the use of mixture of natural compounds.

The one object of the present invention is therefore to provide for the use of at least one natural compound in an amount and for a period of time sufficient to expose numerous benefits within skin care and hygiene care and stabilizing or improving the cosmetic or hygiene compositions or premixes.

It is to be understood that this invention is not limited to the particular compositions, methodology, or protocols described herein. Further, unless defined otherwise, all technical and scientific terms used herein have the same meaning as commonly understood to one of ordinary skill in the art to which this invention belongs. It is also to be understood that the terminology used herein is for the purpose of describing particular embodiments only, and is not intended to limit the scope of the present invention, which will be limited only by the claims.

It is to be understood that. unless otherwise specifically noted, all percentages recited in this specification are by weight.

### BRIEF DESCRIPTION OF THE DRAWINGS

FIG. 1 shows a table illustrating the results in vitro obtained are presented in the following order: the antioxidant capacities as determined by the FRAP, TEAC, and deoxyribose assays. All the samples investigated were found to exhibit anti-oxidative properties.

FIG. 2 depicts UV absorption capacity of Dihydroquercetin (DHQ) and Vitamin C.

FIG. 3 depicts UV absorption capacity of cosmetic crème composition with Dihydroquercetin (DHQ) and Vitamin C.

### DETAILED DESCRIPTION

Molecular biology plays a pivotal role in innovating cosmeceuticals. Ingredient development now begins with the identification of molecular targets. For example, the importance of free radicals in association with skin aging has led in recent years to an intensive search for active substances which eliminate the harmful effects of free radicals and thus protect the tissue from oxidative damage. Skin ageing manifests as age spots, more specifically asmelasma, dyschromia, melanomas, and wrinkling, mainly attributed to free radical damage to the tissues that triggers cross linking and glycation of structural proteins, and pro-inflammatory enzyme systems. The use of flavonoids, in particular, in cosmetics or pharmacy is known per se. Natural antioxidants that quench free radicals are an essential component of anti-ageing formulations. They potentially offer protection against damage to the tissues, and against the detrimental effects of environmental and other agents. Biochemical reactions that accelerate the progression of skin ageing have their roots in inflammatory processes, as inflammation generates micro-scars that develop into blemishes or wrinkles.

Various types of inflammatory mediators may influence melanin synthesis by affecting the proliferation and functioning of melanocytes, pigment-producing skin cells, and normal cutaneous blood circulation. Natural “anti-inflammatory” agents are therefore included in anti-ageing formulations in order to soothe, heal and protect skin tone and integrity.

An increasing amount of scientific evidence supports the beneficial “anti-ageing” effects of several phyto nutrients at the molecular level. For example, plant flavonoids inhibit the age-related atherosclerotic deposits in animals by influencing vascular cell adhesion molecule-1 (VCAM-1) and monocytechemotactic protin-1 (MCP-1) gene expression [10]. Results indicate that Dihydroquercetin (taxifolin), like other flavonoids, has an active role in limiting hair loss, stimulating skin pigmentation, and preserving skin health. The potent bio-activities and relatively low toxicity of Dihydroquercetin (taxifolin) makes it a suitable compound for use in cosmetics [11].

Dihydroquercetin (taxifolin) (or 3,5,7,3′,4′-pentahydroxyflavanone, or (2R,3R)-2-(3,4-dihydroxyphenyl)-3,5,7-trihydroxy-chroman-4-one) occurs in various waste residues of conifer species (Larixcajanderi, Larixdahurica, Larixgmelinii, Larixrussica, Larixsibirica, Pinuspinastersspatlantica) and in Silybummarinum seeds (used for the preparation of the silymarin complex and containing silymarin flavonolignans which are biogenetically formed by oxidative addition of coniferyl alcohol to Dihydroquercetin (taxifolin). It has a chiral bond between cycle B and the two others cycles. Relating to the bioflavonoid group, it possesses a wide spectrum of biological activities [12]. It shows capillary-protecting, anti-inflammatory and gastro-protective action, decreases spasms of sleek muscles of the intestine, increases functions of the liver, possesses anti-radiation protective activity. Dihydroquercetin (taxifolin) has also been shown to have potential applications in reducing skin inflammation [10]. These findings suggest that Dihydroquercetin (taxifolin) is effective for the treatment of atopic dermatitis (AD) by preventing the production of inflammatory cytokines and by reducing skin inflammation [13]. Furthermore, Dihydroquercetin (taxifolin) and its rhamnoside isomers isolated for the first time from kempas could be potent compounds for preventing dental caries [14].

Scientific research has confirmed a wide influence of flavonoid compounds on various levels of the skin. The uppermost layer of the skin, the stratum corneum, is a structure very rich in lipids and other easily oxidizable compounds. In this layer flavonoids can play an efficient role as anti-oxidizing agents and free radical scavengers. Their antioxidant properties enable them to influence deeper, epidermal skin layers, preventing UV radiation damage and inhibiting some enzyme functions. In the dermis, the deepest skin layer, flavonoids influence the permeability and fragility of the micro-vessel system. The valuable features of flavonoids described already makes them priceless for the cosmetic industry. Over the last ten years, extracts containing these compounds have become an integral part of many cosmetic formulations.

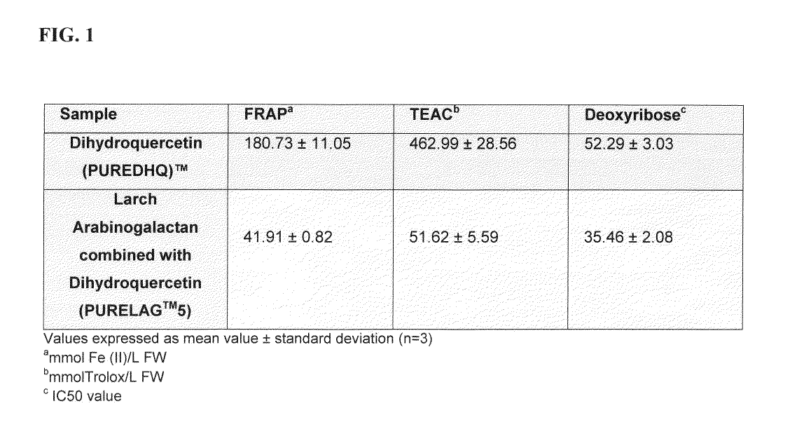

Dihydroquercetin (taxifolin) possess superior antioxidant activity to suppress affects of free radicals [15-21]. It has been shown that Dihydroquercetin (taxifolin) [FIG. 1,2] and phenolic acids have a high UV absorption activity [22,23]. Dihydroquercetin (taxifolin) acts as anti-oxidant with mechanisms involving both free radical scavenging and metal chelation. See FIG. 1. The results shown in FIG. 1 were obtained in vitro and are presented in the following order: the antioxidant capacities as determined by the FRAP, TEAC, and deoxyribose assays. All the samples investigated were found to exhibit antioxidative properties. The FRAP assay takes advantage of electron-transfer reactions. Herein, a ferric salt, Fe(III)(TPTZ)₂Cl₃ (TPTZ=2,4,6-tripyridyl-s-triazine), is used as an oxidant. The reaction detects species with redox potentials <0.7 V [the redox potential of Fe(III)(TPTZ)₂], so FRAP is a reasonable screen for the ability to maintain redox status in cells or tissues. Reducing power appears to be related to the degree of hydroxylation and extent of conjugation in flavonoids. However, FRAP actually measures only the reducing capability based on ferric iron, which is not relevant to antioxidant activity mechanistically and physiologically. The TEAC assay is based on the formation of ferrylmyoglobin radical (from reaction of metmyoglobin with H₂O₂), which may then react with ABTS [2,2′-azinobis(3-ethylbenzothiazoline-6)-sulfonic acid] to produce the ABTS*⁺ radical. ABTS*⁺ is intensively colored, and AC is measured as the ability of the test species to decrease the color by reacting directly with the ABTS*⁺ radical. Results of test species are expressed relative to Trolox. Deoxyribose assays: Hydroxyl radicals, generated by reaction of an iron-EDTA complex with H₂O₂ in the presence of ascorbic acid, attack deoxyribose to form products that, upon heating with thiobarbituric acid at low pH, yield a pink chromogen. Added hydroxyl radical “scavengers” compete with deoxyribose for the hydroxyl radicals produced and diminish chromogen formation. A rate constant for reaction of the scavenger with hydroxyl radical can be deduced from the inhibition of color formation. For a wide range of compounds, rate constants obtained in this way are similar to those determined by pulse radiolysis. It is suggested that the deoxyribose assay is a simple and cheap alternative to pulse radiolysis for determination of rate constants for reaction of most biological molecules with hydroxyl radicals.

Indeed, excess levels of metal cations of iron, zinc and copper in the human body can promote the generation of free radicals and contribute to the oxidative damage of cell membranes and cellular DNA: by forming complexes with these reactive metal ions, they can reduce their absorption and reactivity. It has to be underlined that though most flavonoids chelate Fe2+, there are large differences in the chelating activity. In particular, the Dihydroquercetin (taxifolin) chelates more efficiently Fe2+ than the corresponding flavonoid quercetine [24]. It have been demonstrated in numerous studies in vitro and ex vivo that Dihydroquercetin (taxifolin) inhibits lipid peroxidation, a process that often leads to atherosclerosis [25-27]. In an animal study, Dihydroquercetin (taxifolin) inhibited the peroxidation of serum and liver lipids following exposure to toxic ionizing radiation [28]. Dihydroquercetin (taxifolin)'s inhibitory effects on lipid peroxidation are enhanced by both vitamin C and vitamin E [29].

Recent research findings lend credence to the fact that metabolism, gene expression, and ageing intersect at the molecular level. Nuclear receptors sense a variety of environmental triggers, including dietary components and steroid hormones, and influence metabolic and the ageing process. An increasing amount of scientific evidence supports the beneficial “anti-ageing” effects of several phytonutrients at the molecular level.

For example, Dihydroquercetin (taxifolin) can modulate the expression of several genes, including those coding for detoxification enzymes, cell cycle regulatory proteins, growth factors, and DNA repair proteins. Dihydroquercetin (taxifolin) significantly activates Antioxidant Response Element. ARE (Antioxidant Response Element) in the promoter region of the human NQO1 gene contains AP-1 or AP-1-like DNA binding sites, and AP-1 proteins have been implicated in the formation or function of this and other ARE complexes. Also, ARE-binding proteins in inducing cerebral MT-1 expression and implicates MT-1 as one of the early detoxifying genes in an endogenous defense response to cerebral ischemia and reperfusion [30,31].

One of the important ways in which Dihydroquercetin (taxifolin) may limit the cytokines plain is by preventing elevation of oxidized glutathione concentration and the oxidized/reduced glutathione ratio induced by inflammatory cytokines [32].

Dihydroquercetin (taxifolin) prevents calcium influx, the last step in the cell death process. By inducing the expression of antioxidant defense enzymes, it has the potential to have long-lasting effects on cellular function. This, in turn, could be highly beneficial to cells exposed to chronic oxidative stress [33]. Dihydroquercetin (taxifolin) processes benefit results in both intracellular and extracellular environments. Studies in erythrocytes, mast cells, leucocytes, macrophages and hepatocytes have shown that Dihydroquercetin (taxifolin) renders cell membranes more resistant to lesions. Dihydroquercetin (taxifolin) protects the inner walls of the blood vessels and capillaries against destructive enzymes, decay and free radical damage [34].

Dihydroquercetin (taxifolin) had been evaluated by different studies as the small-molecule regulator of signaling cascades as promising anti-inflammatory agent with biological targets such as COX-2. and related pro-inflammatory mediators (cytokines and chemokines, interleukins [ILs], tumor necrosis factor [TNF]-α, migration inhibition factor [MIF], interferon [IFN]-γ and matrix metalloproteinases [MMPs]) implicated in uncontrolled, destructive inflammatory reaction. Dihydroquercetin (taxifolin) was effective with relevant biological targets that include nuclear transcription factor (NF-κB), p38 mitogen-activated protein kinases (MAPK) and Janus protein tyrosine kinases and signal transducers and activators of transcription (JAK/STAT) signaling pathways has received growing attention [35-37]. Dihydroquercetin (taxifolin) had a significant inhibitory effect on the production of cytokines, formation of ROS and NO, and change in intracellular Ca2+ levels in dendritic cells of bone marrow and spleen [38]. Dihydroquercetin (taxifolin) was attributed to its inhibitory effects on tyrosinase enzymatic activity, despite its effects on increasing tyrosinase protein levels [39].

Studies indicate that Dihydroquercetin (taxifolin) is highly safe and efficacious. In fact, research suggests that dihydroquercetin is even safer than its nutritional cousin, quercetin [40,41]. No toxic effects were observed in rats that were treated with high levels of dihydroquercetin for long periods of time [42-49].

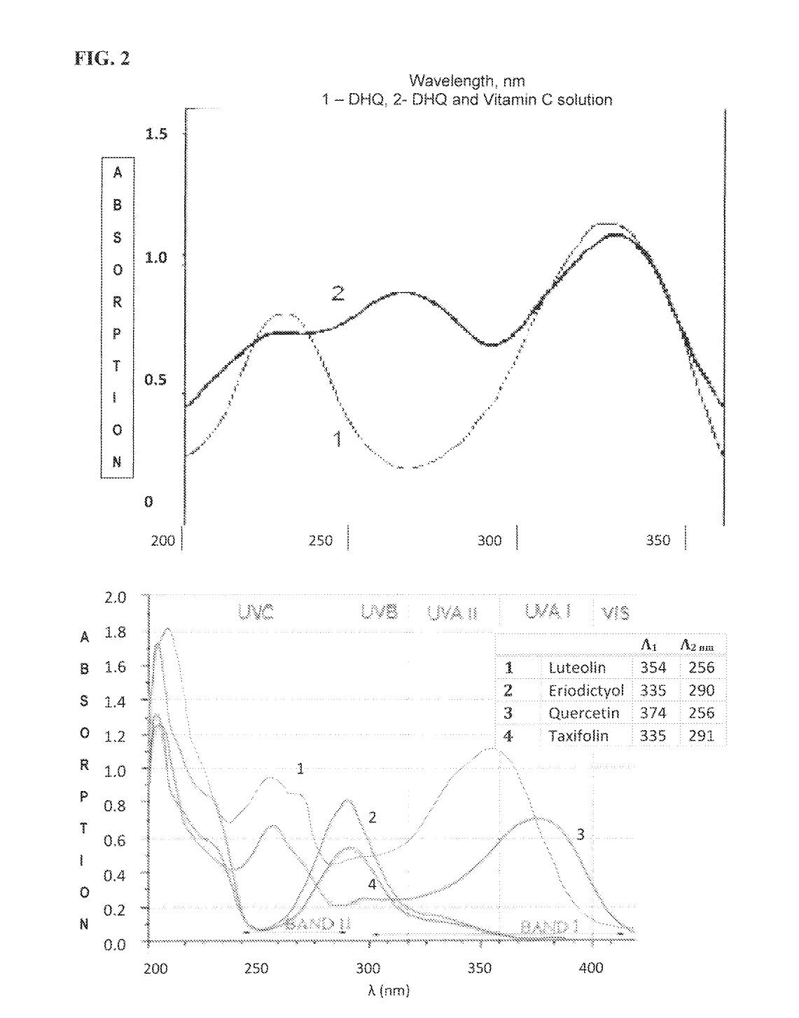

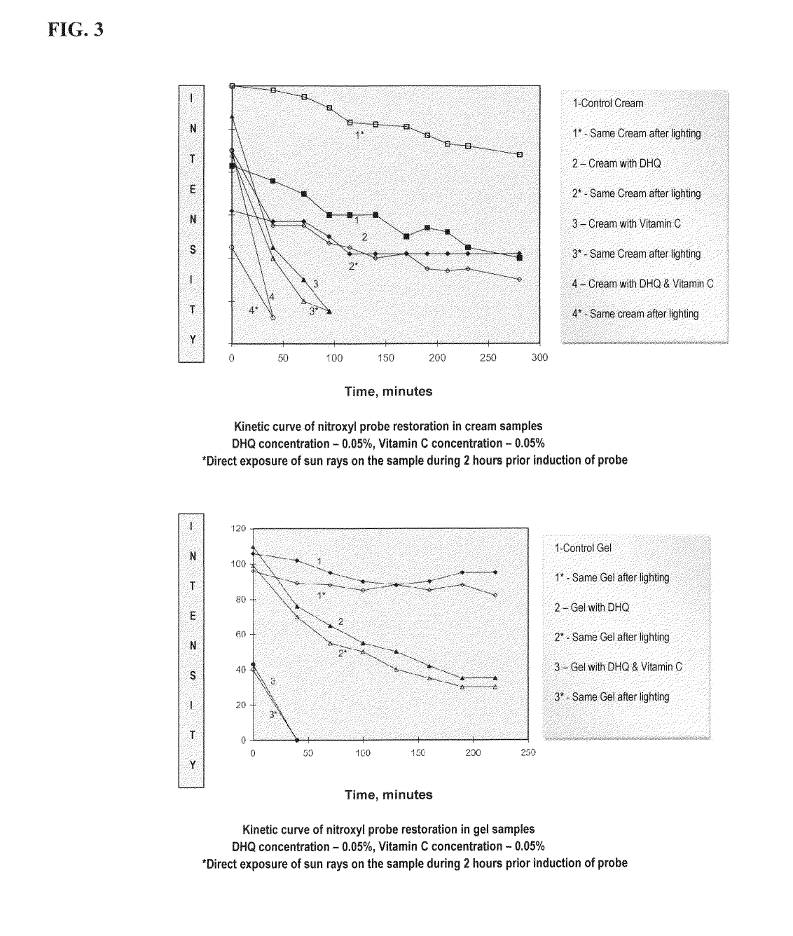

As shown in FIG. 2, a mixture of Dihydroquercetin (DHQ) and Vitamin C (1:1) has advantages over DHQ alone. Vitamin C has λmax 265 nm, which compensates for λmin of DHQ. It is also important that vitamin C at a certain level stabilizes DHQ, preventing its rapid inactivation by UV light and free radicals. Additionally, these compounds act synergetically against free radicals to prevent the damage of the skin and prolong the shelf life of the cosmetic formulation. DHQ protects vitamin C proven capability to stimulate collagen growth in the dermis. As further illustrated in FIG. 3, DHQ & Vitamin C solution features:
Inhibition of hyalutonidase activity;
Lysosome stabilization;
Collagen formation and stabilization;
Participation in metal ion exchanging;
Protection from oxidation damage by ROS;
Decreasing of capillaries toxicosis evidence under the treatment by anticoagulants, salicylic acid and its derivatives:
Vaso-strengthening effect;
Anti-tumoral effect;
Phytoestragenic activity.

Preferred natural compound Dihydroquercetin (taxifolin) is extracted from plant materials from the Larix genus. For example, Dihydroquercetin (taxifolin) is one preferred natural compound because it is found in reasonable commercial yield from Larixcajanderi, Larixczekanowskii, Larixdahurica, Larixgmelinii, Larixkamtschatica, Larixrussica, Larixsibirica, Larixsukaczewii species, which also contain Arabinogalactan, a preferred polysaccharide.

Due to their low aqueous solubility, the use of flavonoids such as Dihydroquercetin (taxifolin) in cosmetic and\or hygiene preparations requires adapted and specific formulations. Since these formulations must also satisfy the constraints associated with their final usage, the compromise between acceptability, concentration and stability is often difficult to reach.

Higher arabinogalactan content often goes hand in hand with higher amount of flavonoid substances, in particular with Dihydroquercetin (taxifolin) [50]. More water soluble forms of flavonoids such as the combination of a Dihydroquercetin (taxifolin) and Arabinogalactan, wherein the polysaccharide Arabinogalactan can be defined as a fiber containing significant amounts of natural antioxidants, mainly Dihydroquercetin (taxifolin) associated naturally to the fiber matrix to serve as a natural pool of nutrients and growth factors that support skin and oral health, wherein Arabinogalactan is a suitable polymer, which is also nonionic, water-soluble or water-dispersible polymer.

Water-soluble Arabinogalactan is a typical useful surface active agent is disclosed above in the context of the bioavailability-enhancing agent that includes a solubilizing agent. Surface active agents generally are an important aspect of the cosmetic and oral compositions, as they can function as surfactants, emulsifiers, foam modulators, and/or active ingredient dispersion agents. Their selection for compatibility with the active ingredient constituents is important. Suitable surface active agents, include those that were discussed in the context of the bioavailability/solubility enhancing agent above, are those which are reasonably stable and foam throughout a wide pH range.

Larch tree extract Arabinogalactan and Arabinogalactan combined with Dihydroquercetin (taxifolin) provides moisture, elasticity, and radiance to skin that boosts collagen production and prevents skin ageing by ‘locking in’ moisture and creating a natural plumping effect. Both Arabinogalactan and Arabinogalactan combined with Dihydroquercetin (taxifolin) provide many desirable skin and oral care effects, and by itself was shown, under clinical conditions, to reduce the appearance of fine lines and wrinkles. It is also proven to provide anti-oxidative properties to help protect skin against UV-induced oxidative damage.

Arabinogalactan has a number of benefits as compared with other polysaccharide polymers. Arabinogalactan is water-soluble, occurs naturally with a narrow molecular weight distribution. While not wishing to be bound by any particular theory, it is believed that because Arabinogalactan is highly branched it is not subject to viscosity problems, as compared to other polymers. Arabinogalactan also stabilizes emulsions. It has been observed in photomicrographs of oil-in-water systems containing Arabinogalactan, the oil-in-water emulsion can be characterized as having smaller and more uniform oil droplets. The ability of Arabinogalactan to produce smaller, more uniform droplets tends to enhance the stability of Arabinogalactan-containing systems over time and is generally known to enhance performance properties. These emulsions have application in cosmetic, personal care, food and industrial applications.

Studies have shown Arabinogalactan to have numerous benefits within skincare, however the object of present invention to use preferably Arabinogalactan combined with Dihydroquercetin (taxifolin) for the purposes already mentioned above and to use for the purposes described more fully below.

Skin Cell Renewal Efficacy.

Skin keratinocytes in the lower level of the epidermis undergo mitosis. These newly formed cells gradually push the existing cells upwards. The older keratinocytes are eventually pushed to the surface where they are sloughed off. Through surface exfoliation, these dead skin cells are removed to reveal younger skin underneath. A higher rate of skin exfoliation may indicate faster cell turnover rates, and a faster cell turnover rate can lead to a reduction in fine lines and wrinkles. Conducted clinical studies into Arabinogalactan properties show Arabinogalactanas both a primary exfoliant as well as its role as an exfoliant enhancer, and in both cases Arabinogalactan displayed significant properties as an exfoliant and, when used in combination with lactose, properties as an exfoliant enhancer (by acting as a film-former, and increasing the functionality of the lactic acid by holding it to the skin).

Reduction of Fine Lines and Wrinkles.

Faster skin cell turnover, as evidenced by enhanced exfoliation, may help explain Arabinogalactan's effect on fine lines and wrinkles, by bringing younger skin to the surface more rapidly. International Resources Inc conducted a study using 15 panelists to evaluate the effect of Arabinogalactan on fine lines and wrinkles in the crow's feet area of the face. In an 8 week, full face, randomized, double-blind, positive-controlled study, Arabinogalactan was proven to reduce fine lines and wrinkles by 19%. Product performance was assessed using both trained evaluators and instrumentally (using silicon replicas with subsequent image analysis) [51].

Reduction of Trans-Epidermal Water Loss (TEWL).

TEWL is the measurement of the water loss from a body that passes through the skin epidermis through diffusion, which then evaporates into the atmosphere. This measure is used to define skin barrier characteristics. Reduction of TEWL (i.e. lower TEWL readings) indicates that the skin barrier is more effective in retaining moisture in the skin, allowing it to feel more moisturized. A test conducted by International Research Services Inc in Port Chester, N.Y., on 21 subjects which measured TEWL after one application of a product containing 2% Arabinogalactan as against a placebo showed a statistically significant reduction in TEWL levels after only 2 and 4 hours, indicating that Arabinogalactan successfully helped to maintain skin barrier function.

Film Forming and Skin Tightening.

Use of the compounds in accordance with the present invention yields an instant skin radiance, as well as having been shown to increase cell metabolic activity by increasing ATP production.

Increased Dispersion of UV Filters.

Arabinogalactan has been shown to improve dispersion of inorganic sunscreen particles (such as titanium dioxide) leading to a more uniform and effective transference onto the skin surface. This leads to less clumping of the sunscreen particles and therefore more efficient packing of the sun protection per UV level.

As used herein, an Arabinogalactan is defined as the class of long, densely branched low and high-molecular polysaccharides with molecular weight range 3,000-120,000. Arabinogalactan consist of a main chain of b-D-(1fi3)-galactopyranose units (b-D-(1fi3)-Galp) where most of the main-chain units carry a side chain on C-6 [fi3,6)-Galp-(1fi]. Almost half of these side chains are b-D-(1fi6)-Galp dimers, and about a quarter are single Galp units. The rest contain three or more units. Arabinose is present both in the pyranose (Arap) and furanose (Araf) forms, attached to the side chains as arabinobiosyl groups [b-L-Arap-(1fi3)-LAraf-(1fi] or as terminal a-L-Araf e.g. a single L-arabinofuranose unit or 3-O-(β-L-arabinopyranosyl)-α-L-arabinofuranosyl units [88-91]. As used herein, “Arabinogalactan” includes purified as well as impure extracts of larch wood and other sources of arabinogalactan.

The conifers are an important source of diterpenoids making one third of neutral wood oleoresin. The main components of the neutral substances of larch wood oleoresin are bicyclic compounds deterpenes or deterpenoids with the labdane structure: epimanool (˜15%), and larixol (˜40%) and its monoacetate (larixylacetate ˜28%). A variety of biological activities have been associated with labdanediterpenes including antibacterial, antifungal, antiprotozoal, enzyme induction, anti-inflammatory modulation of immune cell functions, as well as cytotoxic and cytostatic effects against human leukemic cell lines [52]. In addition to the (antimicrobial, enzyme and endocrine related) properties mentioned above, it is interesting that many labdane type diterpenes also exhibit significant properties against cancer cells.

After the group separation of the larch wood oleoresin, 12.5% of monoterpene hydrocarbons, 0.75% of sesquiterpenes, 18% of diterpene hydrocarbons andaldehydes, 13.5% of diterpene alcohols, and 32.5% of resin acids were obtained. The qualitative and quantitative analysis of the monoterpenes established that the monoterpene fraction contained: a-pinene(20.5%), camphene (0.3%), 8-pinene (23.2%), 3-carene (49.8%), myrcene (0.3%). limonene(1.1%), and 8-phellandrene (2.3%). In the sesquiterpenoid fraction we identified 16 compounds: cyclosativene, longicyclene, alfa-longipinene, sibirene, longifolene, y-elemene, a, y and e-murolenes, beta-selinene, d-, y-, and e-cadinenes, a-humulene, calamenene, and the methyl ether ofthymol, the main components being delta- and gamma-cadinenes and longifolene. From the fraction of diterpene hydrocarbons and aldehydes presented by dehydroabietane, a mixture of dehydroabietinal and abietinaland palustral. By chromatography of the diterpene alcohols were determined epimanool, larixyl acetate, and larixol. The analytical GLC of the mixture of resin acid methyl esters showed the presence in them of the esters of the acids palustric and (or) levopimaric (7.2%), isopimaric (86.2%), dehydroabietic (2.0%). abietic (4.6%), and neoabietic (traces).

A desirable extractant will comprise a compound, or a mixture of compounds, characterized in that the oleoresin (which term as used herein incorporates portions of oleoresin present which are desired to be removed from the source material) is soluble in the extractant. Preferably, the solubility of the oleoresin in the extractant exceeds its solubility in water at the temperatures at which the process is carried out. The oleoresin-rich product stream recovered from the extraction/stripping comprises a useful source of the desired oleoresin fraction. The extractant can be evaporated away, leaving a concentrated oleoresin fraction which can be used as such in the formulation of products such as, for example, personal care cosmetic products. However, this product stream can also be treated as is, or following further concentration or even complete removal of the extractant, to recover any particularly desired component fraction or compound.

It had been known and recorded in the early history of medical practice that natural products such as oleoresins appeared to have some beneficial effects when applied to a variety of human ailments. Ancient remedies in the form of liniments, salves, poultices and tonics often had contained an ingredient such as turpentine, balsam tar, pine tar, rosin, gum resins, and the like. Because such ingredients tended to irritate the skin, the ingredient was employed in small quantities and in a highly diluted state. The early U.S. patent literature reports a number of preparations containing oleoresins which are recommended for relief of all manner of human ailments. Some of the preparations include upwards of ten to twenty ingredients.

U.S. Pat. No. 3,943,248 describes a liniment containing terpentine and pine tar, and recommends its use for reducing skin inflammation. U.S. Pat. No. 238,507 describes a liniment containing terpentine and pine tar for treatment of all types of skin injury including burns. The ointment of U.S. Pat. No. 247,479 contains terpentine and rosin, and is intended for the cure of scab in sheep. The salve of U.S. Pat. No. 256,847 for skin diseases contains rosin as one ingredient. The poultice of U.S. Pat. No. 308,243 contains pine tar and is recommended for felons, carbuncles and abscesses. U.S. Pat. No. 1,426,002 describes a salve containing rosin for skin treatment, and U.S. Pat. No. 2,361,756 proposes a pine tar ointment for general use in skin disorders.

The present invention in the early stages of development involved a program of testing a selected groups of larch oleoresin as health benefit agents in cosmetic and hygiene compositions. It was discovered that concentrated solutions of deterpenoids and rosin acids when applied to traumas of the skin and underlying tissue, rather than irritate and exacerbate the wound area, the solution promoted rapid healing without development of scar tissue. The study of the dermatological application of concentrated oleoresin solutions was extended to include treatment of a broad variety of traumatic and degenerative skin disorders. There was consistent evidence that oleoresin groups of compounds (i.e., abietic acid derivatives), can act as an unusually effective therapeutic agent in the treatment of skin injuries. Burns, ulcers, infections, abrasions and wounds were treated with concentrated solutions of oleoresin. The wide potential of resin acids as bioactive agents gave rise to a growing effort in the search for new applications of the natural forms and their derivatives. In some of these compounds, the antimicrobial activity is associated to the presence in the molecules of functional groups such as the hydroxyl, aldehyde, and ketone or to their cis or trans configurations. The resin acid family covers a spectrum of antimicrobial activities against several microorganisms, from bacteria to fungi [54]. Concentrations of 25-35 weight percent of resin acids in olive oil were applied as a treatment of lymphangitis-cellulitis, small and large abscesses, carbuncles, adenitis of the inguinal and auxiliary lymph glands, phlebitis, and a variety of ulcers including varicose, traumatic, indolent, arteriosclerotic, decubitus and diabetic ulcers. A 15 weight percent resin acids in olive oil terminated pain and infection in the otitis media without mastoid involvement.

Another class of natural actives that support skin hydration are the natural long chain alcohols, such as epimanool, larixyl acetate, and larixol, derived from neutral part of larch oleoresin.

The body's early defense in response to trauma, inflammation or infection, the acute phase response (APR), is a complex set of systemic reactions seen shortly after exposure to a triggering event. The APR is induced by protein hormones called cytokines acting as messengers between the local site of injury and the hepatocytes synthesizing the acute phase proteins such as serum amyloid A. Most cytokines have multiple sources, targets and multiple functions. The pro-inflammatory cytokines can be divided into two major groups with respect to acute phase proteins inductions, the Interleukin-1 (IL-1) type (including Tumor Necrosis Factor-a, TNF-a) and the IL-6 type cytokines (including IL-6 cytokine). These cytokines are secreted primarily by monocytes activated by bacterial toxins or in response to local tissue injury. Larixyl acetate proved to be highly active against Leukotriene Biosynthesis (LT) biosynthesis. The abietane-type diterpenedehydroabietinol showed high LT formation inhibitory activity. Isopimaric acid proved to be a potent inhibitor of 5-LOX mediated LT biosynthesis. The LT biosynthesis inhibitory potential of palustric acid was less pronounced, however, this compound also possessed moderate COX-2 inhibitory activity. Some of these diterpenes are known to possess antimicrobial, anti-ulcer and cardiovascular activities.

The preferred larch wood oleoresin in form of oil or resin is combined with Dihydroquercetin (taxifolin) naturally as one extract according technological process known per se. However, larch wood oleoresin in form of oil or resin can be synergistically enriched with Dihydroquercetin (taxifolin) to contain latter up to 30%.

Larch wood extracts, particularly flavonoid Dihydroquercetin (taxifolin) or polysaccharide Arabinogalactan or Arabinogalactan combined with Dihydroquercetin (taxifolin) or wood oleoresin comprising oil or resin may be incorporated into cosmetic and\or hygiene compositions in an amount from about 0.001% to about 30%, or in an amount from about 0.01% to about 10%.

Further preferred combinations of embodiments are disclosed in the claims.

The advantages of the method of the present invention over pre-existing methods appears clearly from the previous descriptions and embodiments. The present invention describes new original larch wood extracts, particularly extracts from Larixcajanderi, Larixczekanowskii, Larixdahurica, Larixgmelinii, Larixkamtschatica, Larixrussica, Larixsibirica, Larixsukaczewii species. New types of cosmetic and hygiene products are constantly being developed, and new raw materials are adding to the cosmetic and hygiene chemist's selection of personal care ingredients in particularly larch wood extracts Dihydroquercetin (taxifolin), polysaccharide Arabinogalactan, Arabinogalactan combined with Dihydroquercetin (taxifolin), wood oleoresin comprising oil and resin. These larch wood extracts described in the present invention can easily be incorporated in a large panel of cosmetic and hygiene products.

These active larch wood extracts which (i) have as little irritation potential as possible for the skin, hair, oral cavity (ii) have a high free radical-deactivating and anti-inflammatory effects and (iii) have a potential of bioavailability-enhancing agents (iv) are also suitable for the preparation of cosmetic and/or hygiene formulations or preparations.

These active larch wood extracts and compositions comprising these active extracts have a free radical-decomposing effect and can be used for avoiding or reducing skin and/or hair and\or oral damage caused by said free radicals.

These active larch wood extracts can stabilize oxidation-sensitive cosmetically and hygiene active substances and prolong the stability of such formulations.

These active larch wood extracts can possess strong anti-inflammatory activity in cosmetic and hygiene compositions.

These active larch wood extracts are suitable for the use in cosmetic and hygiene formulations or compositions as bioavailability-enhancing agents that includes a solubilizing agents and/or surface active agents, which generally are an important aspect of the cosmetic and hygiene compositions, as they can function as stabilizers, surfactants, emulsifiers, foam modulators, and/or active ingredient dispersion agents.

These active larch wood extracts provide many desirable skin, hair, oral care effects. Of course, it should be understood that a wide range of changes and modifications can be made to the embodiments described above. It is intended, therefore, that the foregoing description illustrates rather than limits this invention, and that it is the following claims, including all equivalents, that define this invention.

What is claimed is:

1. Method of using a natural compound in a cosmetic or hygiene product, the method comprising the steps of:
extracting a larch wood extract from one of a Larix genus plant including Larixcajanderi, Larixczekanowskii, Larixdahurica, Larixgmelinii, Larixkamtschatica, Larixrussica, Larixsibirica and Larixsukaczewiiin; and
manufacturing a cosmetic or a hygiene product by combining the larch wood extract with cosmetically or hygienically active substances to provide a desirable skin, hair or oral care effect.

2. The method according to claim 1. wherein the larch wood extract comprises a flavonoid dihydroquercetin (taxifolin).

3. The method according to claim 1, wherein the larch wood extract comprises a polysaccharide arabinogalactan.

4. The method according to claim 1, wherein the larch wood extract comprises a polysaccharide arabinogalactan combined with a flavonoid dihydroquercetin (taxifolin).

5. The method according to claim 1, wherein the larch wood extract comprises a larch oleoresin.

6. The method according to claim 1, wherein the larch wood extract comprises a larch oleoresin and a dihydroquercetin (taxifolin).

7. A cosmetic or hygiene composition comprising:
a larch wood extract extracted from one of a Larix genus plant including Larixcajanderi, Larixczekanowskii, Larixdahurica, Larixgmelinii, Larixkamtschatica, Larixrussica, Larixsibirica and Larixsukaczewiiin in an amount from about 0.001% to about 30%; and
cosmetically or hygienically active substances.

8. The cosmetic or hygiene composition according to claim 7, wherein the amount of the larch wood extract is from about 0.01% to about 10%.

9. The cosmetic or hygiene composition according to claim 7, wherein said composition has a free radical-decomposing effect such that said composition reduces at least one of a skin damage, a hair damage and an oral damage caused by free radicals.

10. The cosmetic or hygiene composition according to claim 7, wherein said larch wood extract provides stabilization of oxidation-sensitive said cosmetically and hygienically active substances and prolongs stability of said composition.

11. The cosmetic or hygiene composition according to claim 7, wherein said composition possesses strong anti-inflammatory activity.

12. The cosmetic or hygiene composition according to claim 7. wherein said composition is utilized as a bioavailability-enhancing agent.

13. The cosmetic or hygiene composition according to claim 7, further comprising a vitamin C.
